(19) United States
(12) Patent Application Publication
(10) Pub. No.: US 2010/0311835 A1
(43) Pub. Date: Dec. 9, 2010

(54) CIS-9, TRANS-11 ISOMER OF CONJUGATED LINOLEIC ACID (CLA) FOR TREATING INFLAMMATORY DISEASES

(75) Inventors: Doris Bell, Duesseldorf (DE); Albrecht Weiss, Langenfeld (DE); Albert Strube, Neuss (DE); Bernd Fabry, Korschenbroich (DE); Gerhard Jahreis, Jena (DE); Anke Jaudszus, Mechterstaedt (DE)

Correspondence Address: FOX ROTHSCHILD LLP, 997 Lenox Drive, Bldg. #3, Lawrenceville, NJ, 08648, US

(73) Assignee: COGNIS IP MANAGEMENT GMBH, Duesseldorf (DE)

(21) Appl. No.: 12/701,971

(22) Filed: Feb. 8, 2010

Related U.S. Application Data
(62) Division of application No. 10/565,135, filed on Dec. 18, 2006, now abandoned.

(30) Foreign Application Priority Data
Jul. 18, 2003 (DE) 10332712.6

(51) Int. Cl. A61K 31/201; A61P 11/06; A61P 11/00

(52) U.S. Cl. 514/560

(57) ABSTRACT

A composition and medicament comprising the cis-9, trans-11 isomer of conjugated linoleic acid for treating inflammatory diseases, including asthma, is provided. A method for inhibiting the proliferation of human or animal cells of the immune systems involved in an immune response including administering the cis-9, trans-11 isomer of conjugated linoleic acid to a patient is also provided. A nutritional, functional food, or dietary supplement including the cis-9, trans-11 isomer of conjugated linoleic acid is also provided.

### CROSS REFERENCE TO RELATED APPLICATIONS

This application is a divisional of application Ser. No. 10/565,135 filed on Dec. 18, 2006 which is a 35 U.S.C. §371 filing of PCT application PCT/EP2003/014592, filed Dec. 19, 2003, which claims priority from German Application No. 103 32 712.6, filed on Jul. 18, 2003, the entire specifications of which are incorporated by reference herein.

### FIELD OF THE INVENTION

The application relates generally to pharmaceuticals for treating inflammatory diseases, and more particularly to medicaments for treating inflammatory diseases, for example, respiratory diseases including asthma.

### BACKGROUND OF THE INVENTION

Asthma bronchiale is an inflammatory respiratory disease which is accompanied by an increased sensibility of the respiratory channels for various stimuli and a reversible bronchial constriction. The airway epithelium is the first tissue layer to encounter environmental stimuli such as inhaled allergens or microbes and plays a pivotal role in the inflammatory network seen in airways during asthma attacks. The evolution of asthma is primarily based on the inflammation of the bronchial mucous membrane, where mast cells, T-lymphocytes, granulocytes, and inflammation mediators like, e.g., histamine are considered to be seriously involved. Upon exposure to a stimulant, the epithelial cells by themselves will express and release a variety of inflammatory mediators which act in a paracrine, autocrine or endocrine fashion to propagate the disease development. Increased IL-6 and IL-8 production has been reported in airway epithelial cells from patients with bronchial asthma. Recently it has been demonstrated that activation of the nuclear peroxisome proliferator-activated receptor-γ (PPARγ), which is constitutively expressed in bronchial epithelial cells, dramatically inhibits production of inflammatory mediators, suggesting that PPARγ may act as a negative immunomodulator in the airways by protecting non-lymphoid tissue from cytokine-mediated events associated with immune stimulation. Similar to other steroid hormone receptors, PPARγ requires activation by a ligand in order to modulate gene expression by interacting with specific DNA response elements located upstream of responsive genes encoding for inflammatory mediators.

People suffering from asthma show a bronchial hypersensitivity in the early phase of disease which means that the bronchial system reacts with contraction and an overproduction of slime even in case of rather weak and usually neutral stimuli. The dominant role in the pathogenesis of the asthma bronchiale is a spontaneous reaction of type I which is mediated by antibodies of the IgE type. Said antibodies recognise specific allergenic compounds as invaders, form complexes with said allergens in order to stimulate mast cells to degranulate and to emit messenger compounds like, e.g., histamine. These so-called mediators start a chain of reaction and effect an endobronchial obstruction by
bronchial spasm (spasmodic contraction of the medial and small respiratory channels),
swelling of the mucous membrane and inflammatory infiltration of the mucous membrane, and
overproduction of viscous slime in the respiratory system.

As a result the respiratory system becomes blocked and the patient suffers from not getting enough breath. Besides the IgE-mediated spontaneous Type I reaction which usually occurs after a couple of minutes after the stimulation, further allergic reactions are possible even after some hours. It is known that some patients show both types of reaction. Although usually one allergen is responsible for an attack in the beginning of an allergic asthma, however, in the course of the disease other allergenic factors may become important, too, so that a prophylaxis by eliminating potential allergenic factors becomes rather difficult.

Besides bronchial dilatarding agents, asthma therapy uses anti-inflammatory medicaments:
Glucocorticosteroids show anti-inflammatory, anti-allergic, and immune suppressive effects, increase the mucociliar clearance and inhibit the production of inflammation mediators. Further, they readjust the sensibility of the beta-receptors of the respiratory systems for beta-sympathomimetica. Unfortunately, the compounds need hours to become effective, even if applied intravenously, so that they are fully useless in case of an acute attack. In addition, glucocorticosteroids are know for negative side effect, for example an increased glucose concentration in the serum, unwanted fat deposition in the tissue, osteoporosis and skin atrophie.
By means of antihistaminica the messenger histamine which is responsible for allergic reactions is neutralised and emitted in the course of an asthma attack. Antihistaminica inhibit the expression of histamines by the mast cells of the mammal by stabilising them. As a result, the swelling of the mucous membranes goes down, accompanied by an anti-inflammatory effect. Unfortunately, these medicaments are only useful for prophylaxis but unsuitable for fighting a spontaneous asthma attack.

Therefore, it has been an object of the present invention, particularly with respect to the increasing number of resistances and the need for increasing the number of technical means, to identify new compounds which simultaneously inhibit cell proliferation and expression of cytokines, and which show an anti-inflammatory effect in order to fight diseases of the respiratory system and to develop a new medicament which is useful to prevent and to fight spontaneous asthma attacks or at least spend relief, mainly during the sleeping phase.

### SUMMARY OF THE INVENTION

Briefly described, in one aspect of the invention, a medicament comprising the cis-9, trans-11 isomer of conjugated linoleic acid for treating inflammatory diseases is provided. The medicament may act against the hypersensitivity of the bronchia. The inflammatory disease may be a respiratory disease, including a rheumatic disease. The rheumatic disease may include asthma.

In another aspect of the invention, a method for inhibiting the proliferation of human or animal cells of the immune systems involved in an immune response includes administering the cis-9, trans-11 isomer of conjugated linoleic acid to a patient in need thereof. A technical grade of the cis-9, trans-11 isomer of conjugated linoleic acid may be administered, and which includes an amount of at least 30% by weight based on the total weight of the conjugated linoleic acid. The acyl moiety of the technical grade cis-9, trans-11 isomer of conjugated linoleic acid includes at most 30% by weight of trans-10,cis-12 isomers, and in total less than 1% by weight of 8,10-11,13 and trans,trans isomers based on the total content of conjugated linoleic acid. The cis-9, trans-11 isomer of conjugated linoleic acid may be administered sub-cutaneously, intramuscularly, per inhalation, per infusionem, topically or orally to a patient in need thereof, and may also be incorporated into a pharmaceutically acceptable carrier

In another aspect of the invention, a composition for the treatment of asthma includes the cis-9, trans-11 isomer of conjugated linoleic acid.

In yet another aspect of the invention, a nutritional, functional food, or dietary supplement includes the cis-9, trans-11 isomer of conjugated linoleic acid, and may be administered in amounts of from 0.05 to 5 grams per day.

### BRIEF DESCRIPTION OF THE DRAWINGS

FIGS. 1A and 1B illustrate the modulation of LPS/serum-stimulated BEAS-2B by cis-9, trans-11 CLA and LA;

FIGS. 2A and 2B illustrate the effects of cis-9, trans-11 CLA and LA on the production of IL-8 and IL-6 by stimulated BEAS-2B after twenty-four hours; and

FIG. 3 illustrates the expression of IL-8 and IL-6 in LPS and serum-stimulated BEAS-2B over a four hour and twelve hour periods of treatment.

### DETAILED DESCRIPTION OF THE INVENTION

According to an aspect of the invention, the cis-9, trans-11 isomer of conjugated linoleic acid for the production of a medicament for treating inflammatory diseases and for a nutritional, functional food or dietary supplemental agent is provided.

Surprisingly it has been found in vitro that the cis-9,trans-11 isomer of conjugated linoleic acid—compared to other CLA isomers in general and, more specifically, compared to non-conjugated linoleic acid—
(i) inhibits cell proliferation predominantly of epithelial cells and mainly of bronchial epithelial cells for up to 30%,
(ii) support of apoptosis of eosinophilic granuloctyes, and
(iii) reduced the expression of pro-inflammatory cytokines like IL-6 for about 70 and of IL-8 for about 50% (compared to a placebo).

The inhibition of cell proliferation, particularly the proliferation of cells of the immune systems involved in immune response and specific cytokine expression, preferably within cells of the bronchial system or the cartilage tissue of the joints, are accompanied by an anti-inflammatory effect which one can use for fighting
hypersensitivity of the bronchia,
respiratory diseases, particularly asthma, and
rheumatic diseases, particularly rheumatic arthritis,
   but also for reducing the symptoms of inflammation induced diseases in general, like e.g. rheumatic pains, Alzheimer's disease, arteriosclerosis, morbus crohn, colitis ulcerosa and the like.

### Conjugated Linoleic Acid

Conjugated linoleic acid (CLA) represents a commercially available process which usually is obtained by base-catalysed isomerisation of sunflower oil or their respective alkyl esters and subsequent isomerisation in the presence of enzymes. CLA is an acronym used for positional and geometric isomers deriving from the essential fatty acid linoleic acid (LA, cis-9,cis-12-octadecadienoic acid, 18:2n-6). Soon after its initial isolation from grilled ground beef in 1987, CLA was found to exercise pleiotropic beneficial effects spanning from chemoprotection of carcinogenesis, atherosclerosis, and diabetes to inflammation. CLA has several structural and functional properties that are different from those of all-cis-nonconjugated polyunsaturated fatty acids. Namely, the non-methylene interrupted double bond system seems to be decisive for modulating cellular processes that might lead to the observed effects. Emerging evidence has indicated that individual CLA isomers act differently in the biological systems and contribute differently in their beneficial or potential side effects. The cis-9,trans-11 CLA-isomer was most efficacious in inhibiting the growth of cancer cell lines in vitro and in vivo. In contrast, trans-10,cis-12 CLA was observed to have a greater impact on lipid metabolism and adipogenesis. Recently, several studies have demonstrated the ability of CLA to reduce levels of pro-inflammatory eicosanoids such as prostaglandins of the 2-series. Until now, however, there is no specific way to predict the properties of certain isomers, therefore, the identification of new applications and uses is still a question of trial and error. From a physiological point of view the use of the cis-9,trans-11 isomer according to the present invention is of special importance having at least 30, preferably at least 50 and most preferably at least 80% b.w. of said cis-9,trans-11 isomer—calculated on the total CLA content of the crude mixture. In addition, it has been found advantageous if the content of the trans-10,cis-12 isomer is at most 45, preferably at most 10% b.w. and most preferably is less than 1% b.w., and the sum of 8,10-, 11,13- and trans,trans-isomers in total is less than 1% b.w.—again calculated on the total CLA content. Such products can be found in the market for example under the trademark Tonalin CLA-80 (Cognis). Usually, the daily dosage of CLA is 0.05 to 5, preferably 2.0 to 4 g/day.

### Form of Application

cis-9,trans-11 CLA or a respective medicament comprising said CLA isomer are usually applied either sub-cutaneously, intramuscularly, per inhalation, per infusionem or orally—the latter for example in form of a dietary supplement, e.g. in form of a liquid composition or a capsule—or inhaled as a spray. Beside this, a topical application—especially for fighting rheumatic pains—is also possible.

### EXAMPLES

The invention is further illustrated by the following examples, which are not intended to limit the scope thereof.

### Materials and Methods

Cell cultures. The transformed human bronchial epithelial cell line BEAS-2B (Clonetics Cell Systems, St. Katharinen, Germany) and the normal human bronchial epithelial cell line in primary culture (American Type Culture Collection, Manassas, Va., USA) were cultured in T-25-tissue flasks in bronchial/tracheal epithelial cell basal medium (BEBM) supplemented with a variety of growth factors including bovine pituitary extract (0,052 mg/mL), human recombinant epidermal growth factor (0.5 ng/mL), hydrocortisone (0.5 μg/mL), epinephrine (0.5 μg/mL), transferrin (0.01 mg/mL), insulin (5 μg/mL), retinoic acid (0.1 ng/mL), triiodthyronine (6.5 ng/mL), gentamicin (0.05 mg/mL) and amphotericin (0.05 μg/mL), all purchased from Clonetics Cell Systems (St. Katharinen, Germany). Cultures were held at 37° C. in a humidified atmosphere of 5% CO₂ in air and the medium was changed every other day.
Stimulation of cells and fatty acid supplementation. Cis-9,trans-11-CLA and linoleic acid (LA) were obtained from Matreya Inc. (Pleasant Gap, Pa., USA). Purified fatty acids in oil form were dissolved in 96% ethanol to give a 20-mM stock solution, which was further diluted in growth medium to produce the range of test concentrations (20 μg/mL, 10 μg/mL and 5 μg/mL CLA and LA, respectively). At confluence, the cells were detached by exposure to trypsin/EDTA solution (0.05/0.02% in Ca²⁺- and Mg²⁺-free phosphate buffered saline; Biochrom AG, Krefeld, Germany) and reseeded in 12-well plates at a concentration of 1×10⁵ cells/well and cultured for 24 h to allow the cells to attach to the substratum. The medium was then replaced and a final volume of 1 ml of each CLA- and LA-test concentrations, supplemented with 5 μg LPS/mL (lipopolysaccharide, E-coli serotype 026:B6, Sigma, Taufkirchen, Germany) and 10% serum from allergic donors, was added to the wells. A similar volume of fatty acid-free medium containing the indicated stimuli and 0.4% ethanol, corresponding to the max. volume in experimental wells, was used in stimulated control. 1 ml of fresh growth medium containing 0.4% ethanol without any fatty acid and stimuli underwent the same measures of preparation and represented the unstimulated control. After incubation for 24 h at 37° C., supernatants were harvested, separated from non-adherent cells and cell debris by centrifugation (1300 rpm, 10 min), and kept at −20° C. until cytokine analysis. Cells were trypsinised to determine cell numbers and allow quantification of differences in cell proliferation in response to different treatments.
Measurement of IL-6 and IL-8 in cell culture supernatants. IL-6 and IL-8 production of epithelial cells were estimated in duplicate by enzyme-linked immunosorbent assay using commercially available ELISA kits (Pharmingen, Heidelberg, Germany) according to the manufacturer's guidelines. Standard curves and blank buffer were included in duplicate in each assay. When necessary, samples were diluted to fit into the assay range. Specific interleukin concentrations were expressed as picograms of interleukin per millilitre.
Estimation of cytokine gene expression with RT-PCR. Cultures of BEAS-2B and NHBE at an initial density of 1×10⁵ cells/well were stimulated as described above and treated with cis-9,trans-11-CLA or LA in the highest concentration (20 μg/mL) for 4 h or 12 h before total cellular RNA was isolated for cytokine gene expression analysis by reverse transcriptase-polymerase chain reaction. Total RNA preparations were performed by DNase treatment according to the standard protocol of the High-Pure™ RNA isolation kit from Roche (Mannheim, Germany). For RT-PCR analysis, first strand cDNA was synthesized from extracted RNA using random primers, reagents and conditions supplied in the ProSTAR™ First-Strand RT-PCR kit from Stratagene (Amsterdam, Netherlands). One-tenth of the synthesized cDNA was subjected to PCRamplification in a total volume of 25 μl. The primers for IL-6 and IL-8 DNA amplification were designed from published sequences as follows:

| | |
|---|---|
| IL-6 primer | 5′-CCCAGTACCCCCAGGAGAAGAT-3′ |
| forward: | |
| and | |
| reverse: | 5′-CTGCGCAGAATGAGATGA-GTTGTC-3′, |
| IL-8 primer | 5′-CTTGGCAGCCTTCCTGATTT-3′ |
| forward: | |
| and | |
| reverse: | 5′CTCAGCCCTCTTCAAAAACT-3′, |

and gave PCR products of expected sizes of 507 by and 200 bp, respectively. Primer sequences for the housekeeping gene cyclophilin as internal control were: forward 5′-CATCTGCACTGCCAAGACTG-3′ and reverse 5′-CTGCAATCCAGCTAGGCATG-3′, defining a 326 by DNA fragment. The PCR conditions were as follows: initial denaturation at 95° C. for 5 min to activate the AmpliTaq DNA polymerase (Roche, Mannheim, Germany), followed by 30 cycles of 95° C. for 1 min, annealing for 1 min, extension at 72° C. for 1 min, and a final 10 min elongation step at 72° C. Annealing for the IL-6 primer pair was performed at 57° C., for the IL-8 primer pair at 58° C. and for that of cyclophilin at 60° C. PCR reaction products were separated by flat bed electrophoresis in 1.5% agarose gels (Roche, Mannheim, Germany), stained with ethidium bromide, recorded using a geldocumentation-system and analysed densitometrically with Phoretix 1D Advanced Software (Biostep, Jahnsdorf, Germany).
Statistical analysis. To determine significant differences among the treatment groups, the data were evaluated with the Wilcoxon matched-pairs signed-ranks test using SPSS software (version 10.07) and are reported as the means±SEM of at least three independent experiments, each done in duplicate. Significance of difference was set at p<0.05.

### Example 1a

### cis-9,trans-11-CLA Prevented Stimulus-Induced Increase of Proliferation

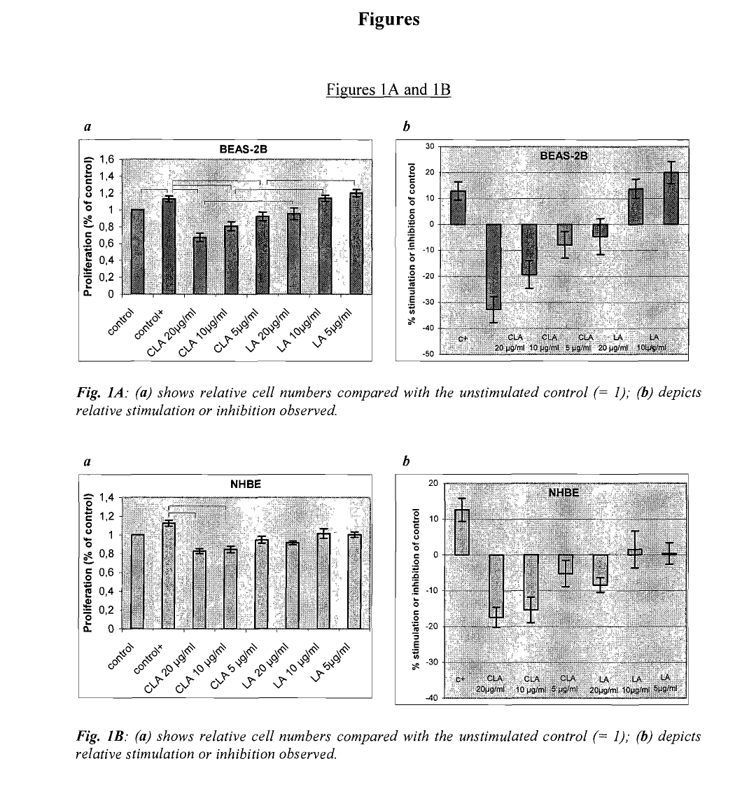

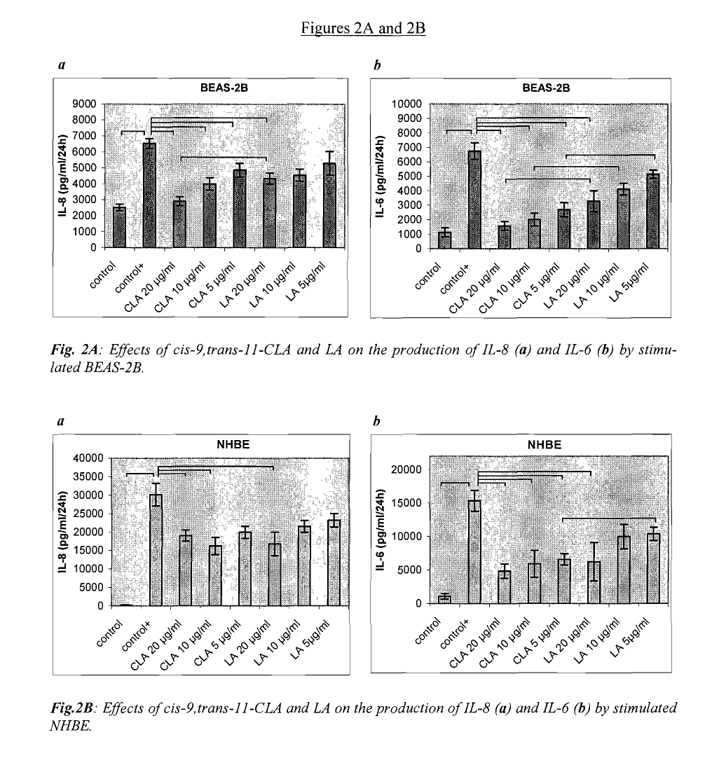

To establish the effects of cis-9,trans-11-CLA in comparison to LA on proliferation of stimulated normal (NHBE) and transformed human bronchial epithelial cells (BEAS-2B), cells grown in medium containing 5 μg LPS/mL and 10% serum from allergic donors and supplemented with either cis-9,trans-11-CLA or LA at different concentrations were harvested after 24 h and cell numbers determined. Exposure of cells to the indicated stimuli without any fatty acid (control+) caused a markedly enhanced proliferation compared with the unstimulated control in both BEAS-2B (by 12.9±3.5%) and NHBE (by 12.6±3.2%). Incubation with 20 μg/mL, 10 μg/mL and 5 μg/mL cis-9,trans-11-CLA significantly prevented the stimuli-induced increase dose-dependent significantly (p<0.05) in BEAS-2B for all tested concentrations (by 32.7±5.0%, 19.3±5.0% and 7.9±5.1%, respectively) and in NHBE for 20 μg and 10 μg cis-9,trans-11-CLA/mL (by 17.4±2.8% and 15.4±3.6%). Treatment with LA under identical conditions led to a small and not significant inhibition of proliferation after 24 h using the maximal concentration (by 4.7±6.9% in BEAS-2B and 8.4±2.1% in NHBE). At lower concentrations, LA caused cell growth. Modulation of proliferation by the isomer was significantly different from that seen in LA-treated BEAS-2B, for all concentrations. The results are shown in FIGS. 1A and 2B.

### Example 1b

### Modulation of Cytokine Expression by Fatty Acid Treatment

IL-6 and IL-8 secretion was examined by ELISA in culture supernatants of BEAS-2B and NHBE after 24 h of incubation with different concentrations of either cis-9,trans-11-CLA or LA in stimuli-containing medium. Basal cytokine expression in both cell lines was low. However, in the presence of 5 μg LPS and 10 serum secretion of both cytokines IL-6 and IL-8 significantly increased. In BEAS-2B, the basal cytokine expression was approximately 2.6-fold higher for IL-8 and 5.9-fold higher for IL-6 in the presence of the indicated stimuli (FIG. 2A). When cis-9,trans-11-CLA was added to the cells sensitised IL-8 and IL-6 production was dose-dependently inhibited in a range from 25.6±3.6% in lowest, to 55.2±5.2% in highest concentration and from 59.9±7.1% to 76.4±4.4%, respectively, compared with the stimulated control+. Decreased cytokine release was also noticed in the corresponding LA-cultures, but was less clear (by 19.3±8.7% to 33.5±5.0% reduced IL-8 and 23.1±8.9% to 50.2±14.1% reduced IL-6). NHBE cytokine release was massively sensitised by LPS— and serum stimulation. Cis-9,trans-11-CLA was similarly effective in suppressing cytokine secretion as seen for BEAS-2B. Significantly less IL-8 was quantified in cis-9,trans-11-CLA treated cells, whereas 10 μg CLA/mL was most efficacious (by 46.0±8.1% compared to control+). CLA-mediated suppression of IL-6 production was even clearer. CLA added together with stimulating LPS and serum revealed a significant decrease in cytokine release in a dose-dependent manner (ranging from 56.9±4.9% to 68.6±5.3%). LA in highest concentration led to significant reduction in cytokine secretion just as well: IL-8 release was reduced by 43.3±14.3% and IL-6 release by 60.0±16.3. However, comparison of both fatty acids showed significant differences in modulating cytokine expression, The results are shown in FIGS. 2A and 2B.

In Table 1, all results are summarized. The percentage of inhibition of stimuli-induced cytokine release is comparatively higher than that seen for proliferation. By representing production of interleukins/100.000 cells, fatty acid-specific differences become distinct: all tested concentrations of cis-9,trans-11-CLA were more efficacious in inhibiting secretion of IL-6 compared with the corresponding LA-treatments. With regard to IL-8 release, the dose-dependent effects are less clear: in BEAS-2B only the highest concentration of the isomer, and in NHBE the middle and the lowest concentration, had a higher depressing effect on IL-8 production than LA.

TABLE 1
Comparison of percentages of inhibition relating to stimulated proliferation
and IL-6 and IL-8 production after 24-h-treatment with cis-9,trans-11-CLA or
LA and the means of cytokine release/100.000 cells

| % inhibition of | | | | |
|---|---|---|---|---|
| proliferation | IL-6-secretion | IL-8-secretion | pg IL | pg IL |

BEAS-2B

| | | | | | |
|---|---|---|---|---|---|
| CLA 20 μg/mL | 32.7 ± 5.0 | 76.4 ± 4.4 | 55.2 ± 5.2 | 792 ± 152 | 1458 ± 136 |
| CLA 10 μg/mL | 19.3 ± 5.0 | 69.7 ± 7.6 | 38.8 ± 5.2 | 841 ± 184 | 1667 ± 160 |
| CLA 5 μg/mL | 7.9 ± 5.1 | 59.9 ± 7.1 | 25.6 ± 3.6 | 983 ± 175 | 1774 ± 154 |
| LA 20 μg/mL | 4.7 ± 6.9 | 50.2 ± 14.1 | 33.5 ± 4.0 | 1181 ± 256 | 1533 ± 121 |
| LA 10 μg/mL | no effect | 38.4 ± 6.6 | 30.6 ± 3.6 | 1220 ± 120 | 1341 ± 110 |
| LA 5 μg/mL | no effect | 23.1 ± 8.9 | 19.3 ± 8.7 | 1440 ± 78 | 1483 ± 209 |

NHBE

| | | | | | |
|---|---|---|---|---|---|
| CLA 20 μg/mL | 17.4 ± 2.8 | 68.6 ± 5.3 | 35.9 ± 11.4 | 1731 ± 387 | 6793 ± 545 |
| CLA 10 μg/mL | 15.4 ± 3.6 | 62.3 ± 10.2 | 46.0 ± 8.1 | 2062 ± 707 | 5659 ± 812 |
| CLA 5 μg/mL | 5.3 ± 3.7 | 56.9 ± 4.9 | 33.3 ± 8.1 | 2049 ± 262 | 6212 ± 513 |
| LA 20 μg/mL | 8.4 ± 2.1 | 60.0 ± 16.3 | 43.3 ± 14.3 | 2007 ± 924 | 5420 ± 1032 |
| LA 10 μg/mL | no effect | 35.0 ± 8.6 | 27.8 ± 10.8 | 2906 ± 530 | 6252 ± 462 |
| LA 5 μg/mL | no effect | 32.1 ± 5.6 | 22.8 ± 5.2 | 3052 ± 292 | 6820 ± 543 |

### Example 2

### Quantification of IL-6 and IL-8 mRNA

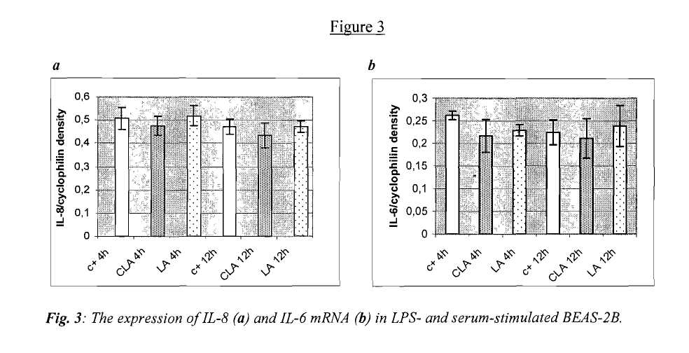

Reverse transcriptase-polymerase chain reaction (RT-PCR) using cyclophilin RNA as internal standard was performed after 4 h and 12 h of incubation of cells without (c+) or with either 20 μg/mL cis-9,trans-11-CLA or LA in stimuli-containing medium. PCR reaction products were separated by flat bed electrophoresis, stained with ethidium bromide, recorded using a geldocumentation-system and analysed densitometrically. The effect of CLA on IL-6 and IL-8 protein levels paralleled mRNA levels. Compared with stimulated control (c+) the mRNA accumulation of both cytokines after the period of stimulation was found to be diminished by the isomer, whereas LA had no effect. Outcomes are presented in FIG. 3.

What is claimed is:

1. A method of preparing a medicament for inhibiting respiratory diseases and/or reducing hypersensitivity of the bronchias, comprising adding to a pharmaceutical base, a technical grade of the cis-9, trans-11 isomer of conjugated linoleic acid (CLA) which comprises at most 30% by weight of trans-10, cis-12 isomers and in total less than 1% by weight of 8,10-; 11,13-; and trans, trans-isomers, based on the total CLA content, wherein administration of an effective amount of said medicament to a person in need thereof provides relief from said respiratory diseases or hypersensitivity of the bronchias.

2. The method of claim 1 wherein said respiratory disease is asthma.


SEQUENCE LISTING (6 sequences)

SEQ ID NO: 1 - DNA, 22 bp, Artificial Sequence
  Primer
cccagtacccccaggagaagat

SEQ ID NO: 2 - DNA, 24 bp, Artificial Sequence
  Primer
ctgcgcagaatgagatgagttgtc

SEQ ID NO: 3 - DNA, 20 bp, Artificial Sequence
  Primer
cttggcagccttcctgattt

SEQ ID NO: 4 - DNA, 20 bp, Artificial Sequence
  Primer
ctcagccctcttcaaaaact

SEQ ID NO: 5 - DNA, 20 bp, Artificial Sequence
  Primer
catctgcactgccaagactg

SEQ ID NO: 6 - DNA, 20 bp, Artificial Sequence
  Primer
ctgcaatccagctaggcatg